(19) United States
(12) Patent Application Publication
(10) Pub. No.: US 2009/0062623 A1
(43) Pub. Date: Mar. 5, 2009

(54) IDENTIFYING POSSIBLE MEDICAL CONDITIONS OF A PATIENT

(75) Inventors: Jason C. Cohen, Appleton, WI (US); Michael Joseph Garvey, Appleton, WI (US); Christopher S. Westra, Appleton, WI (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: KIMBERLY-CLARK WORLDWIDE, INC., Neenah, WI (US)

(21) Appl. No.: 12/108,832

(22) Filed: Apr. 24, 2008

Related U.S. Application Data
(63) Continuation-in-part of application No. 11/847,953, filed on Aug. 30, 2007.

(51) Int. Cl. A61B 5/00

(52) U.S. Cl. 600/300; 705/3

(57) ABSTRACT

Assisting a health care provider in rapidly detecting a possible medical condition in a patient. In an embodiment, a handheld portable device programmed with software instructions is configured to interact with the patient to deduce a list of possible medical conditions. The deduced list of possible medical conditions is provided to a health care provider for diagnosis. Suggested treatment plans may also be provided based on the list of possible medical conditions.

### CROSS-REFERENCE TO RELATED APPLICATION

This application is a continuation-in-part of commonly owned, co-pending U.S. patent application Ser. No. 11/847,953, filed Aug. 30, 2007, the entire disclosure of which is hereby incorporated by reference herein.

### BACKGROUND

Traditionally, when a patient visits a health care provider, a medical application concerning the purpose of the visit is required to be completed. The application typically includes a list of physical attributes and generic health symptoms that are used to gather cursory information regarding the health of the patient. Based on the response to the physical attributes and generic symptoms, a health care provider needs to identify any medical conditions associated with the patient. The health care provider typically relies on training, experience, and medical literature to arrive at a list of potential medical conditions associated with the patient.

This sequential process is monotonous and time-consuming, leaving little time for the provider to focus on actual patient diagnosis to identify the specific medical condition and determine a treatment plan. In addition, the process is subject to human error which could ultimately lead to the wrong diagnosis. Existing systems lack an automated means for assisting health care providers in accurately, rapidly, and consistently identifying a list of potential medical conditions for the patient based on the symptoms expressed by the patient.

### SUMMARY

Embodiments of the invention interact with a patient to identify possible medical conditions of the patient. A decision tree has nodes corresponding to questions relating to the possible medical conditions. Each question is conditionally linked to other questions within the decision tree and leaf nodes represent the medical conditions. Questions from the decision tree are presented to the user, and the decision tree is traversed based on responses received from the user to arrive at one or more of the leaf nodes. The medical conditions associated with the identified leaf nodes are determined and provided to a health care provider to aid the provider in diagnosing the patient.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is an exemplary block diagram of an embodiment of a computing device for use by a patient according to the invention.

FIG. 2 is an exemplary block diagram of an embodiment of various software modules integrated within a computer readable medium according to the present invention.

FIG. 3 is an exemplary flow chart of an embodiment of a decision tree algorithm according to the invention.

FIG. 4 is an exemplary block diagram of a decision tree for traversal in an embodiment of the present invention.

FIG. 5 is an exemplary block diagram of a decision tree that includes some questions randomly selected to identify other medical conditions.

Corresponding reference characters indicate corresponding parts throughout the drawings.

### DETAILED DESCRIPTION

Embodiments of the invention assist a health care provider in rapidly and accurately detecting medical conditions in a patient. The health care provider includes, but is not limited to, a medical practitioner who is assigned to diagnose a medical condition of the patient. In some embodiments, the invention includes a handheld portable device programmed with software instructions configured to interact with the patient to deduce a list of potential medical conditions. The device may also provide a definition of each medical condition and a course of recovery actions to be adopted in treating each medical condition.

Health care providers have access to a multitude of resources to identify potential medical conditions for a given health topic. However, to focus in on the list of medical conditions, information retrieval can be a cumbersome procedure. Sorting through large databases of a specific health related topic often requires research through large volumes of medical literature. Determining the health-related topic(s) for the patient is arduously deduced through the administration of a history and physical for the patient by the health care provider. This typically involves a series of questions and answers relating to the physical attributes and symptoms expressed by the patient. In most instances, this process is time consuming for the health care provider. Further, many patients do not immediately know the answers to some of the questions in part because they are not trained to look at their symptoms from a clinical point of view.

Disclosed in this application is a mechanism for assisting health care providers in determining the potential or possible medical conditions of each patient. In an embodiment, the mechanism includes a device such as a mobile computing device. Further, the patient may not be well versed on his specific medical condition to express his condition clearly. Hence, based on the physical attributes and symptoms experienced and expressed by the patient, the device makes intelligent decisions to focus in on a list of potential medical conditions and to prompt the patient to view any symptoms in an analytical manner. In an embodiment, the definition of each medical condition and a course of recovery actions to be adopted in treating each medical condition are also identified. The information is provided to the health care provider for review. Once further diagnosis on the patient has been performed and the exact medical condition identified or confirmed by the provider, the course of action to be adopted by the patient for recovery may be inputted and/or verified by the health care provider through the device.

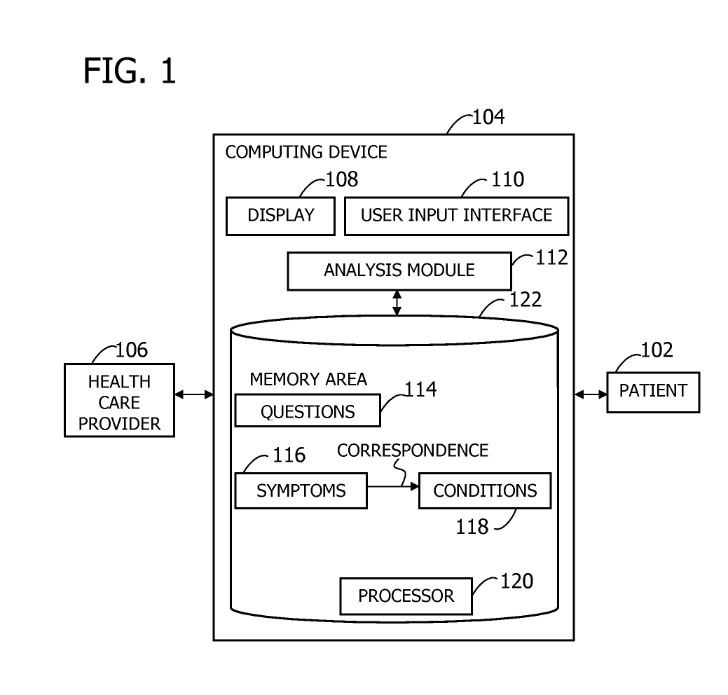

FIG. 1 is a block diagram of a computing device 104 in an embodiment of the invention. Device 104 may be a mobile hand held computing device such as but not limited to a personal digital assistant (PDA) device, a laptop, a wireless device, or cellular phone embodied with decision software. The device 104 includes a processor 120 coupled to an user input interface 110 and a display 108, wherein the display 108 is driven in response to the user input via the user input interface 110. The display 108 includes any display on any device known or contemplated in the art. For example, the display 108 may include a textual display, graphical display, or both. The user input interface 110 may be a keyboard, touch screen, pointer or any other device known in the art through which a user inputs a desired selection to the processor 120. The processor 120 is further coupled to an analysis module 112, and further to a storage means such as a memory area 122. In an exemplary embodiment, the memory area 122 is a portable memory card that is received within the device 104.

In a further embodiment, the device 104 supports audio. The patient 102 may select an option on the device 104 that generates an audio representation of the questionnaire (e.g., each question is read aloud). Such an embodiment is applicable, for example, to vision-impaired patients. In such an embodiment, the device 104 includes a headphone output for connection to a pair of headphones to preserve the privacy of the patient 102. Correspondingly, the device 104 may include a microphone for receiving voice input responses from the patient 102 to the presented questions. In such an embodiment, the device 104 may include voice recognition software for interpreting the received voice input responses and performing actions (e.g., selecting another question) based on the interpretation. In this manner, the patient 102 does not need to type, enter, select, or otherwise use buttons or a stylus on the device 104 to complete the questionnaire. Such an embodiment is useful for patients 102 with arthritis or other joint or muscular ailments that would prevent them from otherwise being able to complete the questionnaire using the device 104.

In an embodiment of the invention, once a patient 102 has checked into a health center, the patient 102 is provided the hand held device 104 to supply information pertaining to the symptoms that the patient 102 is experiencing. The processor 120 may include a reference code for each patient 102 to provide each patient 102 with an identity, such as but not limited to the patient's social security number and a security validation pin code to validate the identity of the patient 102. In an alternate embodiment, a fingerprint sensing device may be coupled to the device 104. When a patient's fingerprint matches that of a stored fingerprint from the memory area 122, the patient 102 may be granted access to the analysis module 112.

When a patient 102 logs into the device 104, any selection input through the processor 120 is stored in the memory area 122. This information may automatically be retrieved when the patient 102 logs into the device 104 during subsequent visits to the health center. This aspect fosters maintaining a record and monitoring the patient's medical history and progress during the course of the affiliation to the health center. The patient's medical record stored in the memory area 122 may further be shared by means of an external network interface, such as but not limited to a network (e.g., the Internet) to an external agency. An exemplary example one such agency may be the insurance company of the patient 102.

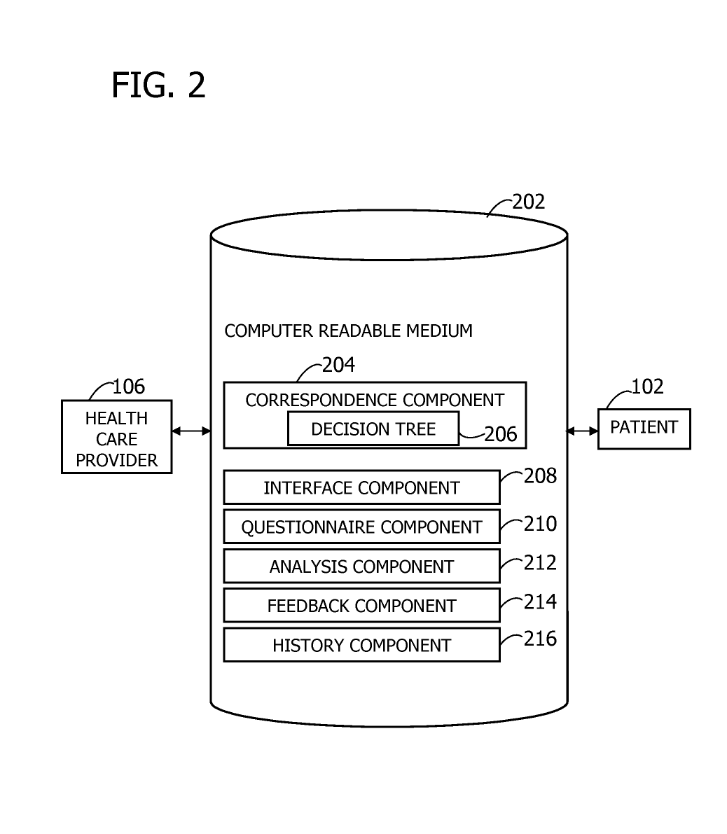

On successfully logging into the device 104 in an embodiment, the processor 120 is configured to display on the display 108 a plurality of health topics that may be presented to the patient 102 for selection. The health topics are broad and may pertain to various bodily disorder terms such as chest inflammation, flu, skin allergy, or other topics that a patient 102 may readily understand and therefore select accurately. The topics may be arranged in a sequential alphabetical order, wherein a specific topic may be selected by the patient 102 from a pull down menu. In an alternate embodiment, health topics may be classified under different alternative categories such as but not limited to disorders of various bodily parts, organs, or specific diseases (e.g., urology, obstetrics, cardiology, etc.). The patient 102 may input his health topic selection to the processor 120 by means of the user input interface 110 via an interface component 208 such as illustrated in FIG. 2. The interface component 208 is stored along with other computer-executable components on one or more computer-readable media such as computer-readable medium 202.

Referring to FIG. 1 and FIG. 2, a block diagram illustrates various software modules integrated within a computer readable medium. Once the health topic pertaining to the patient 102 is selected, the patient 102 is presented with a series of questions 114 in real time by means of a questionnaire component 210 stored in the memory area 122. The analysis module 112 dynamically selects or modifies each question in real time based on the response received from the patient 102 to one or more of the preceding questions.

In an embodiment, the questions 114 presented to a patient 102 may be classified into two categories: preliminary and secondary questions. Preliminary questions presented to a patient 102 are those questions that are directed towards gathering information relating to the physical attributes of a patient 102 such as age, sex, ethnicity, smoking status, etc., so as to present additional relevant questions that pertain to the individual physical attributes of that patient 102. Once the preliminary questions have been answered, the responses are stored in the memory area 122, and may be retrieved when the patient 102 logs into the device 104 during subsequent visits. In an embodiment, questions that require to be updated by the patient 102 regularly (such as smoking status) may be presented once more, during subsequent visits to the health center.

After the preliminary patient questions have been completed, the device 104 presents the patient 102 with secondary questions retrieved from the memory area 122 which relate to the symptoms experienced, and is based on the topic of selection. In an embodiment, the analysis module 112 implements a decision tree 206 via a correspondence component 204, thereby guiding the patient 102 through discrete branch alternatives based on the selections inputted to each question. In an embodiment, different decision trees may exist for different health topics selected, such that different questions 114 may be presented to the patient 102 from the memory area 122 based on the selected health topic.

In a further embodiment of the invention, the patient 102 is presented with four selection options at each decision branch node corresponding to possible answers to the question associated with that node: yes/no/sometimes/unknown. If a patient 102 selects the “unknown” selection option or other response indicating uncertainty, the processor selects and presents a different question to enable focusing in on the patient's medical conditions (e.g., without placing any consideration on the prior question). If a patient 102 selects the “sometimes” selection option, the processor 120 implements, for example, a neural network logic algorithm. Based on this algorithm, a question associated with a deceased tree branch of the decision tree, at least one iteration prior to the present iteration may be presented to the patient 102. Depending on the response selected by the patient 102 to that question, the processor may traverse through the existing decision tree branch or may pursue the path led by the tree branch of the prior iteration(s).

Other selection options are within the scope of aspects of the invention and affect the traversal through the decision tree. For example, other selection options include SOMETIMES/RARELY/NEVER. These options allow other questions (e.g., other tree branches) to be asked to improve the accuracy of the diagnosis and to identify conditions that might otherwise not be identified if only YES/NO options were available. For example, for the question “do you have heartburn,” a YES selection might indicate that an ulcer is the underlying condition whereas a SOMETIMES selection may indicate heartburn as the condition.

On the basis of this combined algorithmic logic, the processor 120 is guided by means of an analysis component 212 through different branches of the decision tree 206 based on the responses provided by the patient 102 to each question. This process is effective in probing into and focusing in on only those domains of a selected medical topic, where there exists a close correlation between the physical attributes and symptoms expressed by the patient 102, and the potential medical conditions. In particular, the memory area 122 stores a correspondence between a plurality of symptoms 116 and the medical conditions 118 associated with the symptoms 116. The analysis and selections may be stored in the memory area 122 and retrieved during the patient's subsequent visits to the health center by means of the history component 216.

In a further embodiment, if a specific selection made by the patient 102 contradicts an earlier selection when a similar question is presented during the course of traverse through the decision tree 206, an analysis may be performed wherein a new question similar to the prior questions is presented to the patient 102, and a selection pertaining to that question is accepted as a true selection. Alternatively or in addition, the health care provider is notified of the contradiction for further evaluation.

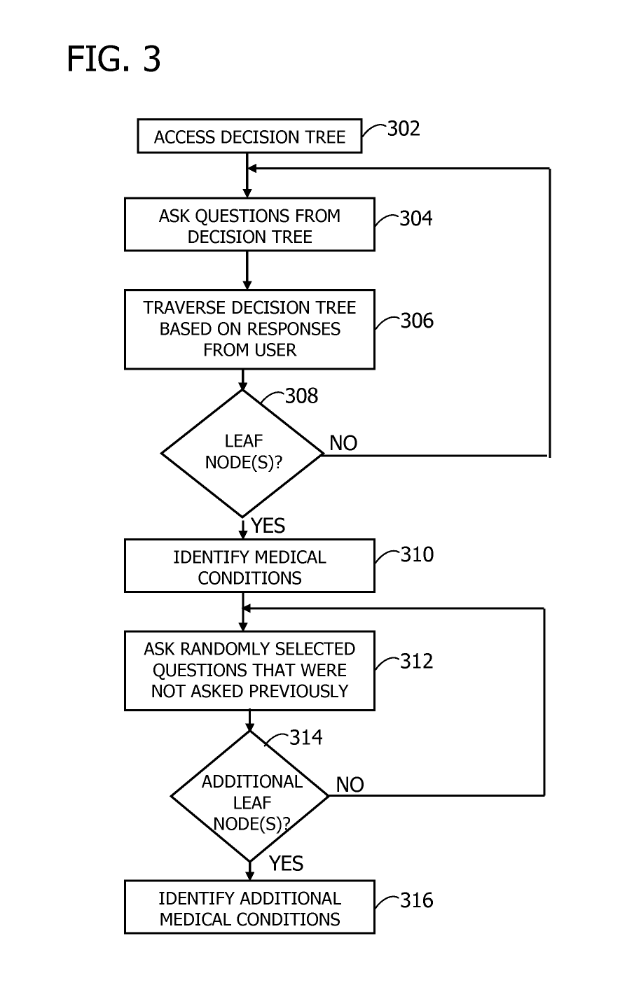

When the processor has focused in sufficiently to arrive at one or more leaf nodes such as shown at 308 in FIG. 3, the medical conditions 118 corresponding to those leaf nodes are identified to the health care provider.

Alternatively or in addition, the analysis module 112 receives vital statistics and other attributes of the patient 102 from another data source. For example, the analysis module receives data from diagnostic equipment. An example of diagnostic equipment that may be used is a blood pressure monitor, pulse monitor, or any other health care equipment that provides a quantitative or qualitative assessment of the patient 102.

The device 104 may be connected to the diagnostic equipment by means such as but not limited to a BLUETOOTH brand network or a hard-wired cable, to input the patient's vital characteristics such as but not limited to blood pressure. In an embodiment, such vital characteristics of the patient 102 may automatically be detected and input in real time into the device 104, or manually selected for input by the health care provider 106 or other health care professional.

Once the vital characteristics of the patient 102 are inputted, the device 104 may proceed to factor the received vital characteristics into the decision tree to affect the resulting list of possible medical conditions 118. This enables the processor 120 to focus in on the exact medical condition of the patient 102.

In a further embodiment of the invention, the patient 102 may be presented with a question from a different node of the decision tree 206. This node is not selected based on any previous analysis in an embodiment, but is rather based on the attributes specified by the patient 102 during the preliminary questions, other factors, or randomly selected. The objective of this exercise is to gather data from the patient 102 pertaining to various other patient potential medical conditions 118 that were not identified previously. Therefore, symptoms that may be experienced by the patient 102, but are not intended to be the purpose of visit to the health center may be elicited to focus in on a different list of potential medical conditions 118. If the patient 102 does not respond affirmatively to these questions, the processor traverses back to the original leaf node of the prior iteration, and the analysis is terminated. Therein, the information provided by the patient 102 is stored within the memory area 122, and the patient 102 is prompted that the potential medical conditions 118 have been determined and will be provided to the health care provider.

Thereafter, the device 104 may present the patient 102 with several options through the display 108. In an embodiment, the display 108 may display an option to present several advertisements of non-prescriptive medications, which allude to the recently analyzed topic selected by the patient 102. Special coupons for such medications may be presented, and may be printed by means of a remote printer via a wireless means such as but not limited to BLUETOOTH brand network, or a hard-wired cable coupled to the device 104. In a further embodiment, the patient 102 may be presented with an option of selecting from a menu a list of entertainment selections that help the patient pass the time spent waiting at the health center.

The patient 102 submits the device 104 to a health care professional, who in turn may present the device 104 to a health care provider 106. The health care provider 106 is provided with an authorized access code and security pin to access the patient's medical history. After successful authentication, the device 104 presents the provider 106 with the potential medical conditions 118 determined by the device 104. Further, the device 104 may also provide the list of questions 114 that were presented to the patient 102, the responses that were elicited by means of the decision tree 206, any other symptoms or warnings associated with the possible medical conditions 118, and/or recommendations for further analysis using other diagnostic equipment.

The health care provider 106 is also provided access to one or more of the following: the list of potential medical conditions 118 associated with the patient 102, the definition of each medical condition, and a course of recovery actions that need to be adopted in treating each medical condition. Therefore, without additional medical analysis and literature review, the health care provider 106 has a clear understanding of the potential medical conditions 118 associated with the patient 102 in a relatively short period of time. As a consequence, valuable time resources that would have otherwise been spent in determining the potential medical conditions 118 of the patient 102 may now be resourcefully invested in diagnosing the specific medical condition, and determining a plan for treating that medical condition.

In a further embodiment of the invention, if the patient 102 has been inconsistent to the questions 114 presented on at least two or more occasions, the device 104 presents the health care provider 106 with the analyzed medical conditions 118 based on the patient's response to the questions 114. In addition to the analyzed medical conditions 118, the device also displays a warning message notifying the health care provider 106 of the decision questions 114 where the patient 102 had been inconsistent in responding, and that the analyzed medical conditions 118 may not be a true representation of the medical conditions associated with the patient 102.

Based on the potential medical conditions 118 presented by the device 104, the health care provider 106 diagnoses the patient 102 with a particular medical condition.

Once the medical condition has been identified, the health care provider 106 may input an option to view through the display 108 a pre-loaded motion film/image of the medical condition such as but not limited to an ultrasound film or an x-ray image. The provider 106 may also extract from the device 104 a definition of the medical condition and specific external references wherein additional information on the medical condition may be obtained, and a list of potential medications that may be prescribed in order to treat that medical condition. In an exemplary embodiment, the prescriptive medication may be an herbal-based drug or a supplement for treating that medical condition. In some embodiments, medications promoted by pharmacies that have a business relationship with the health center may be recommended. In an alternate embodiment, a provision may be made for the provider 106 to enter a professional preference for a specific medication to treat a particular medical condition. This preference may be stored in the memory area 122 by means of the feedback component 214, and presented to that provider 106 for recommended prescription when the same medical condition has been diagnosed in a different patient.

In a further embodiment, a course of recovery actions may be presented to the provider 106 advising of any specific course of action and dietary restrictions that require to be adopted by the patient 102 in treating the medical condition. In an alternate embodiment, based on professional experience, the provider 106 may input a preference of a specific course of action in treating the medical condition. The prescribed medication and personal preferences inputted into the device 104 through the feedback component 214 may be used to influence the questions 114 presented to the patient 102 during subsequent visits, to analyze the effects of the prescribed medication on the medical progress of the patient 102. A series of questions may also be asked based on known side effects of the prescribed medication to ascertain whether the patient 102 is experiencing any of the side effects. Consequently, the processor 120 may also traverse through a different path of the decision tree 206 based on the selections inputted by the patient 102 to those questions 114.

In addition, in the event that the health care provider 106 diagnoses the patient 102 with a medical condition that was not defined in the list of medical conditions 118 described above, the health care provider 106 may input the diagnosed medical condition into the memory area 122 through the feedback component 214 by means of the user input interface 110. The medical condition is used to subsequently influence the questions 114 presented to future patients, thereby modifying the focal node points on the decision tree 206 from where traversal may begin or traverse. Such a feature is useful in analyzing the occurrence of these medical conditions for a different patient.

Once the patient 102 has been discharged from the health center, the patient record contained in the device 104 may be transferred to a primary health center server for the reference of the health center personnel via a wireless means, such as but not limited to a BLUETOOTH brand network, or a hard wired cable coupled to the device 104. When the patient 102 subsequently checks into the health center, the identity of the patient 102 may be verified by a health center professional. Upon validation, the patient data record may be transferred to the device 104 from the health center server before the device 104 is provided to the patient 102.

FIG. 3 illustrates a flow chart of a decision tree 206 indicating an exemplary technique for arriving at a list of potential medical conditions 118 associated with a patient 102. As an example, the patient 102 may be presented the topics of chest inflammation, flu, and skin allergy. Based on the indicated topics, the patient 102 may enter a selection based on a general understanding of the symptoms being experienced.

The decision tree 206 is accessed at 302. Questions 114 from the decision tree 206 are asked at 304. For example, if the patient 102 is visiting the health center for the first time, the patient 102 is presented with preliminary questions wherein the physical attributes of the patient 102 such as age, sex, ethnicity, and smoking status may be input into the processor through the user input interface 110 and stored in the memory area 122. The decision tree 206 contemplates that only relevant questions that pertain to the individual physical attributes of the patient 102 are presented for selection. Once the preliminary questions are answered, the responses are stored in the memory area 122, and may be retrieved when the patient 102 logs into the system during subsequent visits.

When a specific topic is selected by the patient 102, additional questions are presented that relate to the symptoms experienced by the patient 102 with respect to the selected topic. The decision tree 206 is traversed at 306 based on the responses by the patient 102. Depending on the specific responses provided, the patient 102 is presented with dynamically varying questions that probe deeper into additional symptoms or circumstances under which the previously queried symptoms may be experienced. With each progressive question that is dynamically selected based on the answers to one or more previous questions, the decision tree 206 focuses in and iteratively analyzes the responses to the presented questions to arrive at a solution relating to the leaf nodes that further correspond to various potential medical conditions 118 of the patient 102. If the traversal does not arrive at a leaf node at 308, the process continues with questions at 304. After the traversal arrives at a leaf node at 308, the medical conditions 118 corresponding to the leaf node(s) are identified at 310. One or more of the medical conditions 118 may correspond to each of the leaf nodes for the specific symptoms expressed by the patient 102.

Randomly selected questions not previously asked are presented at 312 to attempt to identify other unidentified ailments of the patient 102, and traversal of the decision tree 206 continues. If additional leaf nodes are determined, the medical conditions 118 associated with the additional leaf nodes identified at 316.

In a further embodiment of the invention, any information supplied by the patient 102, or analysis performed on the patient's medical conditions may be stored and retrieved from the memory area 122 to dynamically influence the questions 114 presented to the patient 102 in future visits to the health center. This aspect is effective in determining if the patient's medical condition is influenced by a previously detected medical condition, or is a consequence of the side effects of the medications that were prescribed during earlier visits.

In some embodiments, the device 104 includes the capability to download a plurality of medical topics and the analysis modules associated with each topic into its memory area 122. For example, the device 104 may have a wired or wireless interface that connects to a computer such as a personal computer or a server via a website. The server has access to a plurality of medical topics including the analysis algorithms and decision tree 206 associated with each topic. A user (e.g., health care provider 106) via a user interface may select one or more medical analysis modules such as analysis module 112 and download the selected module into the memory area 122 of the device 104.

In an embodiment, the invention comprises an expert advice website. The server presents at least one webpage accessible via the Internet wherein the webpage includes the user interface. The server receives user input from the user interface, and drives the webpage in responsive to the received user input. Software for the analysis module 112 is stored in a memory accessible by the server and executed by the server. In this embodiment, a user may access the webpage and respond to questions presented by the webpage in order to determine a topic to download into device 104, identify the accompanying analysis algorithms to be provided with the topic, and additional modules relating to the output that are generated through the device 104.

Optionally, the analysis module 112 may be downloadable to a personal computing device for execution by the computing device, or loaded to the computing device for further downloading to the device 104. In yet another optional embodiment the device 104 interfaces with memory cards or a plurality of flash memories. Each memory has the analysis module 112 relating to a different topic and one of the flash memories is connected to the processor 120 for executing the analysis software of the connected flash memory.

In a further embodiment, each health center may have a professional licensing agreement with a service provider of the analysis module 112 to license the device 104 and/or the associated analysis module 112. In addition, the service provider may update the website periodically with new advancements in medical technology. Such updates include but are not limited to efficient optimization algorithms that arrive at a patient's medical condition with minimal iterative questions, as well as increasing the number and size of decision trees associated with expanding medical topics. The professional licensing agreements may differ based on the size and nature of each health center and associated health care providers, and may be mutually negotiated between the provider and the health center management.

In an alternate embodiment, the device 104 is a PDA-based device or a kiosk placed at convenient locations in the proximity of the health center. As described above, the patient 102 may enter an identification number and security pin to log into the health center network server remotely by means of a network such as the Internet. Once the patient 102 has successfully logged in, the process progresses as described herein. After the analysis has been performed and the medical conditions 118 determined, the patient 102 is provided with a printed receipt containing a patient identification code.

The patient 102 may check into the health center at a scheduled appointment time and present the identification code to a health care professional. The health care professional on validating the patient's identity may in turn retrieve the patient's record through a device 104 connected to the health care server by means of a wide area network such as but not limited to the Internet. Thereafter, the healthcare professional may provide the device 104 containing the medical record of the patient 102 to the health care provider 106, and the process progresses as described in the earlier embodiments.

A primary advantage of the device 104 is that it may be used in circumstances in which a patient 102 may not be aware of medical conditions that the patient 102 might have. Based on the decision tree logic that presents intelligent questions in real time, the device 104 may focus in on the most appropriate solution that corresponds to the list of medical conditions. Additionally, device 104 provides a means for editing the decision tree algorithms depending on preferences of the health care provider 106 and the actual medication prescribed for each patient 102 by each health care provider 106.

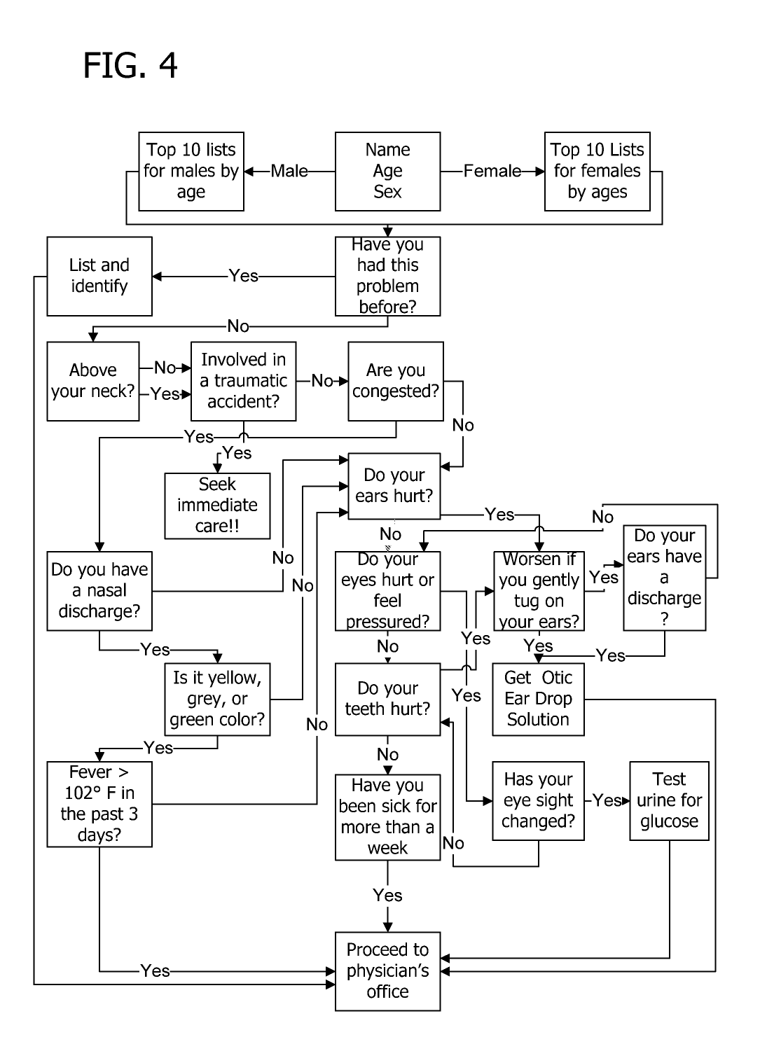

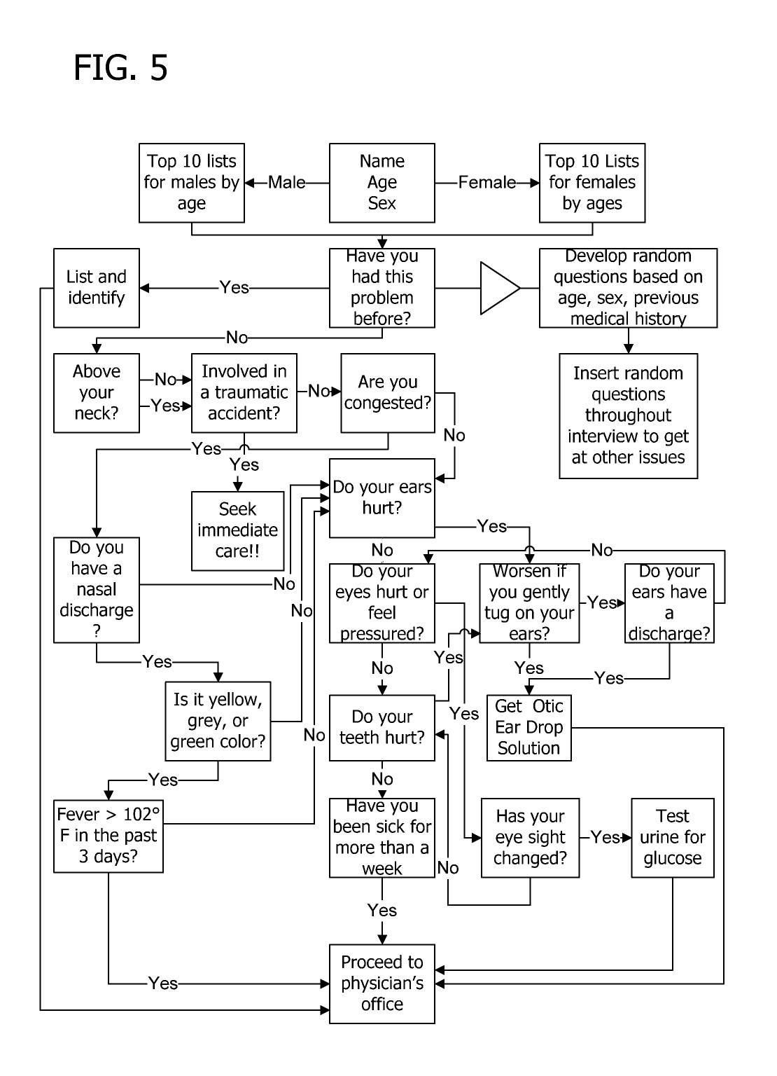

Examples of the decision tree 206 are illustrated in FIG. 4 and FIG. 5. The decision tree in FIG. 4 begins with exemplary preliminary questions about the patient, and then poses exemplary secondary questions about symptoms based on the answers to the preliminary questions. The decision tree in FIG. 5 is similar to the decision tree in FIG. 4, but includes random questions developed to identify other medical conditions (e.g., conditions other than those underlying the symptoms that compelled the patient to seek medical help).

Those skilled in the art will note that the nodes in the decision trees in FIG. 4 and FIG. 5 may be connected to other nodes in a variety of ways based on the answers to the questions. That is, any one particular node may be included in a plurality of different traversal paths through the decision tree.

### Exemplary Operating Environment

A computing device or computer such as described herein has one or more processors or processing units and a system memory. The computer typically has at least some form of computer readable media. Computer readable media, which include both volatile and nonvolatile media, removable and non-removable media, may be any available medium that may be accessed by computer. By way of example and not limitation, computer readable media comprise computer storage media and communication media. Computer storage media include volatile and nonvolatile, removable and non-removable media implemented in any method or technology for storage of information such as computer readable instructions, data structures, program modules or other data. For example, computer storage media include RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computer. Communication media typically embody computer readable instructions, data structures, program modules, or other data in a modulated data signal such as a carrier wave or other transport mechanism and include any information delivery media. Those skilled in the art are familiar with the modulated data signal, which has one or more of its characteristics set or changed in such a manner as to encode information in the signal. Wired media, such as a wired network or direct-wired connection, and wireless media, such as acoustic, RF, infrared, and other wireless media, are examples of communication media. Combinations of any of the above are also included within the scope of computer readable media.

The system memory includes computer storage media in the form of removable and/or non-removable, volatile and/or nonvolatile memory. The computer may operate in a networked environment using logical connections to one or more remote computers, such as a remote computer.

Although described in connection with an exemplary computing system environment, embodiments of the invention are operational with numerous other general purpose or special purpose computing system environments or configurations. The computing system environment is not intended to suggest any limitation as to the scope of use or functionality of any aspect of the invention. Moreover, the computing system environment should not be interpreted as having any dependency or requirement relating to any one or combination of components illustrated in the exemplary operating environment. Examples of well known computing systems, environments, and/or configurations that may be suitable for use with aspects of the invention include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Embodiments of the invention may be described in the general context of computer-executable instructions, such as program modules, executed by one or more computers or other devices. The computer-executable instructions may be organized into one or more computer-executable components or modules. Generally, program modules include, but are not limited to, routines, programs, objects, components, and data structures that perform particular tasks or implement particular abstract data types. Aspects of the invention may be implemented with any number and organization of such components or modules. For example, aspects of the invention are not limited to the specific computer-executable instructions or the specific components or modules illustrated in the figures and described herein. Other embodiments of the invention may include different computer-executable instructions or components having more or less functionality than illustrated and described herein. Aspects of the invention may also be practiced in distributed computing environments where tasks are performed by remote processing devices that are linked through a communications network. In a distributed computing environment, program modules may be located in both local and remote computer storage media including memory storage devices.

In operation, a computer executes computer-executable instructions such as those illustrated in the figures to implement aspects of the invention.

The embodiments illustrated and described herein constitute exemplary means for defining the decision tree, and means for dynamically selecting the series of questions based on input received from the patient.

The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

When introducing elements of aspects of the invention or the embodiments thereof, the articles “a,” “Van,” “the,” and “said” are intended to mean that there are one or more of the elements. The terms “comprising,” “including,” and “having” are intended to be inclusive and mean that there may be additional elements other than the listed elements.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

What is claimed is:

1. A mobile computing apparatus for identifying one or more medical conditions possibly associated with a patient, said mobile computing apparatus comprising:
a display;
a user input interface;
a memory area for storing a plurality of questions related to a plurality of symptoms, said memory area further storing a correspondence between the plurality of symptoms and a plurality of medical conditions; and
a processor configured to execute computer-executable instructions for:
selecting a series of questions from the plurality of questions stored in the memory area;
presenting each of the selected questions to the patient in succession via the display;
receiving, via the user input interface, a response to each of the presented questions as the questions are presented, wherein the response corresponds to one or more of the symptoms stored in the memory area, and wherein each next question in the series is dynamically selected based on the response received from the patient to a previous question; and
determining, based on the correspondence between the plurality of symptoms and the plurality of medical conditions stored in the memory area, one or more of the medical conditions associated with the patient.

2. The mobile computing apparatus of claim 1, wherein the memory area further stores a medical history of the patient, and wherein each next question in the series is dynamically selected based on the response received from the patient to the previous questions and based on the medical history of the patient.

3. The mobile computing apparatus of claim 1, wherein the medical history comprises a list of previously prescribed medications, and wherein the processor is further configured to select the series of questions based on side effects associated with the previously prescribed medications.

4. The mobile computing apparatus of claim 1, wherein the processor is further configured to identify at least one medication or herbal supplement to treat the determined medical conditions.

5. The mobile computing apparatus of claim 1, wherein receiving the response from each of the presented questions comprises receiving a response from the user indicating uncertainty by the user, and wherein the processor is further configured to execute computer-executable instructions for selecting and presenting another series of questions to the user, said other series of questions relating to the uncertainty response.

6. The mobile computing apparatus of claim 1, wherein the processor is further configured to identify medical literature for further information regarding the determined medical conditions.

7. The mobile computing apparatus of claim 1, wherein the processor is further configured to display one or more of the following on the display: advertisements, games, videos, and coupons.

8. The mobile computing apparatus of claim 1, wherein the plurality of questions stored in the memory area are grouped according to a medical topic associated therewith.

9. The mobile computing apparatus of claim 1, wherein the memory area corresponds to a memory card inserted into the mobile computing apparatus.

10. One or more computer-readable media having computer-executable components for aiding in the diagnosis of a patient, said components comprising:
a correspondence component including a decision tree, said decision tree defining a relationship between a plurality of questions and a plurality of medical conditions;
an interface component for receiving input from the patient;
a questionnaire component for dynamically selecting a series of the questions from the questionnaire component based on input received from the patient via the interface component;
an analysis component for identifying one or more of the medical conditions for the patient based on the decision tree in the correspondence component and on the input from the patient received via the interface component; and
a feedback component for receiving a diagnosis for the patient from a health care provider and comparing the received diagnosis with the medical conditions identified by the analysis component, wherein the feedback component adjusts the decision tree stored in the correspondence component based on the comparison.

11. The computer-readable media of claim 10, wherein the correspondence component comprises a plurality of the decision trees each corresponding to one of a plurality of health care providers, and wherein the feedback component adjusts the decision tree for each of the health care providers based on the diagnosis from the corresponding health care provider.

12. The computer-readable media of claim 10, wherein the correspondence component, the interface component, the questionnaire component, the analysis component, and the feedback component are associated with a web service and communicate with a mobile computing device associated with the patient.

13. The computer-readable media of claim 10, wherein the correspondence component, the interface component, the questionnaire component, the analysis component, and the feedback component are associated with one or more of the following: a mobile computing device, a wireless device, a cellular telephone, and a personal digital assistant.

14. The computer-readable media of claim 10, further comprising one or more of the following:
means for defining the decision tree; and
means for dynamically selecting the series of questions based on input received from the patient.

15. The computer-readable media of claim 10, wherein the questionnaire component further selects questions based on interaction data associated with medications currently taken by the patient.

16. The computer-readable media of claim 10, wherein the feedback component provides the adjusted decision tree to a plurality of health care providers.

17. The computer-readable media of claim 10, further comprising a history component for storing the input received by the interface component and the diagnosis received by the feedback component, wherein the history component is specific to the patient.

18. A method comprising:
accessing a decision tree having nodes corresponding to questions, wherein each of the questions is conditionally linked to other questions within the decision tree, said decision tree having leaf nodes representing medical conditions;
applying the questions from the decision tree to the user;
traversing the decision tree based on responses received from the user to the applied questions to identify one or more of the leaf nodes;
determining the medical conditions associated with the identified leaf nodes;
identifying a plurality of questions within the decision tree that were not applied to the user;
applying one or more randomly selected questions from the identified plurality of questions to the user and traversing the decision tree based on responses to the applied randomly selected questions to identify additional medical conditions; and
providing the determined medical conditions and the additional medical conditions to a health care provider.

19. The method of claim 18, further comprising receiving data regarding vital statistics of the user, wherein traversing the decision tree comprising traversing the decision tree based in part on the received data.

20. The method of claim 18, wherein the decision tree corresponds to a type of illness.
